(19) United States
(12) Patent Application Publication
(10) Pub. No.: US 2010/0210919 A1
(43) Pub. Date: Aug. 19, 2010

(54) METHOD AND APPARATUS FOR MONITORING PREDETERMINED PARAMETERS IN A BODY

(76) Inventors: Arie Ariav, D.N. Hof Ashkelon (IL); Vladimir Ravitch, Ashkelon (IL)

Correspondence Address: DANIEL J SWIRSKY, 55 REUVEN ST., BEIT SHEMESH, 99544, IL

(21) Appl. No.: 12/679,658

(22) PCT Filed: Sep. 23, 2008
(86) PCT No.: PCT/IL08/01276

§ 371 Date: Mar. 23, 2010

Related U.S. Application Data
(60) Provisional application No. 60/960,276, filed on Sep. 24, 2007.

(51) Int. Cl. A61B 5/00

(52) U.S. Cl. 600/300

(57) ABSTRACT

A method and apparatus for monitoring a predetermined parameter of interest in a body, includes: applying to the body a sensor including an acoustical transmitter and an acoustical receiver spaced from the transmitter to define between them an acoustical transmission channel which includes a part of the body exhibiting the parameter of interest; and monitoring from a remote location the transit time of an acoustical wave transmitted through the acoustical transmission channel from the acoustical transmitter to the acoustical receiver, the monitored transit time being influenced by the parameter of interest and thereby providing a measurement of the parameter of interest. The method and apparatus are particularly useful for monitoring functions of various organs (e.g. the liver, kidneys, lungs, heart, etc.) in a living body but may also be used in non-medical applications, such as for measuring water seepage in concrete.

### FIELD AND BACKGROUND OF THE INVENTION

The present invention relates to a method, and also to an apparatus, for monitoring predetermined parameters of interest in a body. The invention is particularly useful in medical applications, such as in monitoring the functions of various organs, (e.g., the liver, kidney, lungs, heart, etc.), in a living body, e.g., after a transplant operation or other surgical procedure, and is therefore described below especially with respect to such applications, but it will be appreciated that the invention could be used in other medical, as well as non-medical, applications.

It is frequently necessary or desirable to measure certain parameters of an organ transplant before or during the transplant operation, or continuously after the transplant operation. For example, in a liver transplant operation, it is frequently necessary or desirable to measure the degree of blood saturation of the liver, the temperature of the liver to provide information as to inflammatory processes, and/or the mechanical condition or flexibility of the organ tissues after taking drugs, etc. An example of such a method and apparatus is described in U.S. Pat. No. 5,833,603, which issued Nov. 10, 1998. However, the known techniques for making such measurements generally do not provide real-time, instantly available, information concerning the monitored parameters, and/or at best provide a relatively imprecise measurement of such parameters.

Problems are also involved in monitoring various parameters in non-medical applications, for example, for checking water penetration or the like in concrete building constructions. In many cases, such measurements cannot be made in a real-time manner or without destroying a specimen of the body being examined.

In this respect, reference is made to U.S. Pat. No. 6,447,448, which issued Sep. 10, 2002, and also to applicant's International Application PCT/IL2007/000935, published as International Publication No. WO 2008/012820 A2 on 31 Jan. 200, both disclosing methods and apparatus for measuring various parameters of bones and joints. As distinguished from those disclosures, the invention of the present application is directed to methods and apparatus for measuring various parameters of living body organisms, such as the liver, kidneys, lungs, heart, etc., as well as of other, non-medical bodies, such as concrete bodies for determining water seepage therein.

### OBJECT AND BRIEF SUMMARY OF THE PRESENT INVENTION

An object of the present invention is to provide a method, and also apparatus, for precisely monitoring in real time a predetermined parameter of interest in a body, particularly in an organ of a living body.

According to one aspect of the present invention, there is provided a method of monitoring a predetermined parameter of interest in a body, comprising: applying to the body a sensor including an acoustical transmitter and an acoustical receiver spaced from the transmitter to define between them an acoustical transmission channel which includes a part of the organ of the living body exhibiting the parameter of interest; and monitoring from a remote location the transit time of an acoustical wave transmitted through the acoustical transmission channel from the acoustical transmitter to the acoustical receiver, said monitored transit time being influenced by the parameter of interest and thereby providing a measurement of the parameter of interest.

Preferably, the transit time of the acoustical wave is monitored according to the technique described in our prior U.S. Pat. No. 6,621,278, by changing the frequency of the acoustical wave transmitted by the transmitter to the receiver via the acoustical transmission channel, while maintaining the number of waves in a loop including the acoustical transmission channel as a whole integer irrespective of the changes in the predetermined parameter of interest. As described in the above-cited US patent, such a technique provides a highly precise way of monitoring changes in transit time, and therefore changes in any parameter which affects the transit times by changing the transit velocity and/or the transit distance.

In some described preferred embodiments, the sensor is implanted in the body and includes an RF transmitter for transmitting its measurements to a receiver externally of the body. In other described preferred embodiments, the sensor is a passive unit implanted in the body; and the monitoring of the transit time is effected by an active control unit which is located externally of the body and which includes a wireless coupling for supplying power to the sensor unit, and a wireless coupling feedback circuit for enabling the receiver to control the transmitter of the sensor unit.

The invention is particularly useful for monitoring desired parameters on an organ of a living body during, or after, a surgical procedure.

According to another aspect of the present invention, there is provided a method of monitoring a parameter of interest in a body, comprising: providing the body with a sensor unit including an acoustical transmitter and an acoustical receiver spaced from the transmitter to define between them an acoustical transmission channel which includes a part of the body exhibiting the parameter of interest; and monitoring the transit time of an acoustical wave transmitted through the transmission channel from the transmitter to the receiver by a control unit which includes a feedback circuit from the receiver to maintain the number of waves in a loop including the transmitter as a whole integer irrespective of changes in the parameter of interest; wherein the sensor unit is a passive unit applied to the body; and wherein the monitoring of the transit time is effected by an active control unit which is located externally of the body and which includes a wireless coupling for supplying power to the sensor unit, and a wireless coupling for a feedback circuit for enabling the receiver to control the transmitter of the sensor unit.

According to other aspects of the present invention, there is provided apparatus for monitoring a predetermined parameter of interest in a body, or in an organ of a living body, according to the above methods.

As indicated above, the described method and apparatus are particularly useful for monitoring one or more predetermined parameters of interest in an organ transplanted into a human being. It will be appreciated, however, that the method and apparatus could also be used in other medical applications, e.g., for monitoring various parameters of organs during a conventional surgical operation to inspect or repair a damaged organ. In such a case, the sensor could be mounted on a handle to facilitate manipulation of the sensor during the surgical operation and its removal at the end of the surgical operation.

It will also be appreciated that the method and apparatus could be used for monitoring parameters of interest in non-medical applications, such as in concrete constructions, in a real-time/non-destructive manner.

Further features and advantages of the invention will be apparent from the description below.

### BRIEF DESCRIPTION OF THE DRAWINGS

The invention is herein described, by way of example only, with reference to the accompanying drawings, wherein:

FIG. 1 schematically illustrates one form of method and apparatus constructed in accordance with the present invention for monitoring a predetermined parameter of interest in an organ of a living body, such as the liver;

FIG. 2 is a schematic diagram of a preferred electrical control and monitoring system for use with the method and apparatus of FIG. 1;

FIG. 3 schematically illustrates one construction of the apparatus of FIGS. 1 and 2 for implantation in the body;

FIG. 4 illustrates another construction of the apparatus of FIG. 3 for implantation in the body;

FIG. 5 illustrates a sensor constructed in accordance with the invention for application to the organ to monitor the parameter of interest in the course of a surgical procedure, and then to be removed from the body;

FIG. 6 illustrates another sensor, constituted of a matrix of acoustical transmitters and receivers defining a matrix of acoustical channels;

FIG. 7 is an enlarged fragmentary view more particularly illustrating the construction of the matrix-type sensor of FIG. 6;

FIG. 8 is a block diagram illustrating a passive wireless sensor arrangement, i.e., wherein the sensor does not include a power supply but receives its power in a wireless manner from an externally located active unit;

FIG. 9 illustrates one form of passive wireless sensor including simplex inductive couplings;

FIG. 10 is a block diagram of a passive wireless sensor including simplex optical couplings;

FIG. 11 is a block diagram illustrating a passive wireless sensor including a duplex inductive coupling;

FIG. 12 is a block diagram of a passive wireless sensor including a duplex antisidetone circuit;

FIG. 13 illustrates a passive wireless sensor operating according to the one-port mode;

FIG. 14 is a block diagram illustrating another passive wireless sensor operating according to the one-port mode;

FIG. 15 illustrates an oscillator which may be used in the constructions of FIG. 13 or 14;

FIG. 16 schematically illustrates a passive wireless sensor used for monitoring changes of bone density;

FIG. 17 schematically illustrates a piezoelectric transducer which may be used in any of the above sensors;

FIG. 18 illustrate a passive wireless sensor used for monitoring blood flow; and

FIG. 19 illustrates a system constructed in accordance with the present invention using a plurality of passive wireless sensors.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

### Sensor Units and Control System

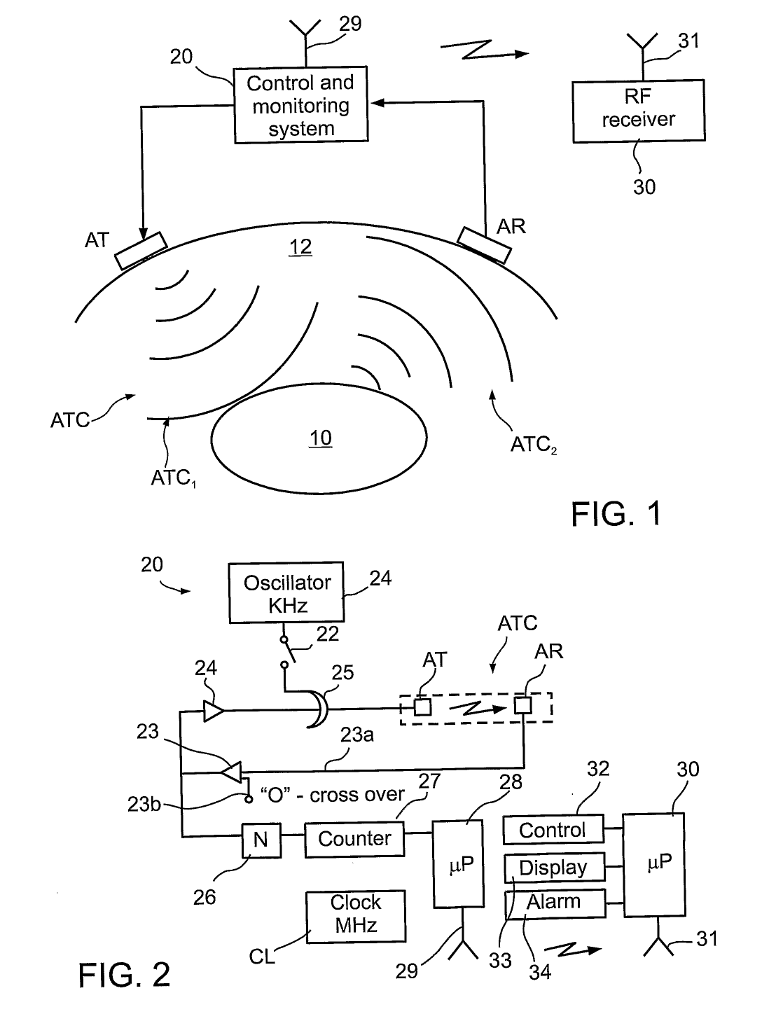

FIG. 1 schematically illustrates an embodiment of the invention for monitoring one or more predetermined parameters of an internal organ 10, commonly referred to as a viscus, in a living body 12. For purposes of example, the viscus 10 may be a liver transplanted into the body 12, whose functioning is to be monitored during the transplant operation and/or continuously after the transplant operation. For example, the parameter(s) monitored may be the degree of liver saturation with blood, changes in temperature which may indicate an inflammatory process, and/or changes in shape or flexibility which may be due to the effects of a drug, etc.

It will be appreciated, however, that the invention could also be used for monitoring predetermined parameters of the natural liver or of other natural internal organs of the body, such as the kidneys, etc. It will be further appreciated that the invention could also be used for monitoring predetermined parameters of interest in other internal organs of the body, such as the kidneys, lung, heart, pancreas, intestines, or in other types of bodies, such as concrete structures.

The apparatus schematically illustrated in FIG. 1 includes an acoustical transmitter AT and an acoustical receiver AR defining between them an acoustical transmission channel ATC which, in this case, includes the direct waves ATC₁ transmitted by transmitter AT towards the liver 10, and the reflected waves ATC₂ reflected by the liver 10 and received by the receiver AR. As will be described more particularly below, the illustrated apparatus includes a control and monitoring system 20 which controls the acoustical transmitter AT and the acoustical receiver AR to change the frequency of transmission of the acoustical waves in accordance with the parameter being monitored. The changes in frequency are such as to maintain the number of waves in a loop, which includes the acoustical transmission channel ATC, as a whole integer irrespective of changes in the parameter of interest. Thus, the changes in frequency produce, in real time, a measurement of the changes in the transit time, and thereby a measurement of changes in any parameter influencing the transit time.

Circuit 20 is preferably constructed according to the above-cited U.S. Pat. No. 6,621,278. Such a circuit is capable of producing a precise measurement of changes in the transit time in the acoustical wave, and therefore a very precise measurement of changes in the parameter(s) of interest influencing such changes in transit time.

In the FIG. 1 embodiment, the implanted system 20 includes an RF transmitter for transmitting its measurements to a receiver, generally designated 30, externally of the body and an antenna 31 for receiving the transmitted measurements.

FIG. 2 more particularly illustrates the construction and operation of the control and monitoring circuit 20 which is implanted in the subject's body, and which transmits its measurements to an RF receiver 30 which is external to the subject's body.

Briefly, the system illustrated in FIG. 2 operates by: (a) transmitting from transmitter AT a cyclically-repeating energy wave through the acoustical transmission channel ATC defined with receiver AR; (b) changing the frequency of the transmission while maintaining the number of waves in the loop including the acoustical transmission channel as a whole integer; and (c) utilizing the changes in frequency of the transmission to provide an indication of the monitored parameter. In the described preferred embodiment, operation (b) includes: detecting a predetermined fiducial point in each cyclically-repeating energy wave received by receiver AR; and continuously changing the frequency of the transmission in accordance with the detected fiducial point of each received energy wave such that the number of energy waves in the loop of the transmission channel is a whole integer.

More particularly, the system illustrated in FIG. 2 operates as follows: Initially, oscillator 21 is energized while switch 22 is closed so as to cause transmitter AT to transmit a succession of sonic pulses until such pulses are received by receiver AR. Once the pulses are received by receiver AR, switch 22 is opened so that the pulses received by receiver AR are thereafter used for controlling the transmitter AT.

As shown in FIG. 2, the sonic signals received by receiver AR are fed to a comparator 23 via its input 23a. Comparator 23 includes a second input 23b connected to a predetermined bias so as to detect a predetermined fiducial or reference point in the received signal. In the example illustrated in FIG. 2, this predetermined fiducial point is the “zero” cross-over point of the received signal; therefore, input 23b of comparator 23 is at a zero bias.

The output of comparator 23 is fed to an amplifier 24, e.g., a monostable oscillator, which is triggered to produce an output signal at each fiducial point (zero cross-over point) in the signals received by receiver AR. The outputs from amplifier 24 are fed via an OR-gate 25 to trigger the transmitter AT for the next sonic pulse. Since switch 22 is open, transmitter AT will thus be triggered by each signal received by the receiver AR to transmit the next sonic pulse in the succession of pulses.

It will thus be seen that the frequency of the output pulses or signals from transmitter AT will change with a change in the velocity, and/or transit distance of the acoustical wave, between the transmitter AT and receiver AR. It will also be seen that the number of wavelengths or pulses in the loop including transmitter AT and receiver AR will be a whole integer. This change in frequency by the transmitter AT, while maintaining the number of waves between the transmitter and receiver AR as a whole integer, enables a precise determination to be made of the transit time, and thereby of the parameter being monitored affecting the velocity and/or transit distance.

A summing circuit, including counter 26, counter 27, clock CL and microprocessor 28, enables the detected frequency difference, and thereby the measurement precision, to be increased by a factor “N”. Thus, the precision of the measurement can be preset, almost without limitation, by the selection of the appropriate frequency, clock rate for clock CL, and summation factor “N” for counter 27.

As further shown in FIG. 2, the output from microprocessor 28 is fed to the antenna 29 of an embedded RF transmitter for transmission to the external receiver 30, via its receiving antenna 31. External receiver 30 may also include a microprocessor for processing the information communicated to it from the embedded sensor, and for providing a measurement of the one or more predetermined parameters of interest. As further shown in FIG. 2, the output from external receiver 30 may be used for control, display, and/or alarm purposes, as schematically shown by blocks 32, 33 and 34.

Further details of the construction and operation of such a monitoring circuit are described in the above-cited U.S. Pat. No. 6,621,278, the contents of which are incorporated herein by reference.

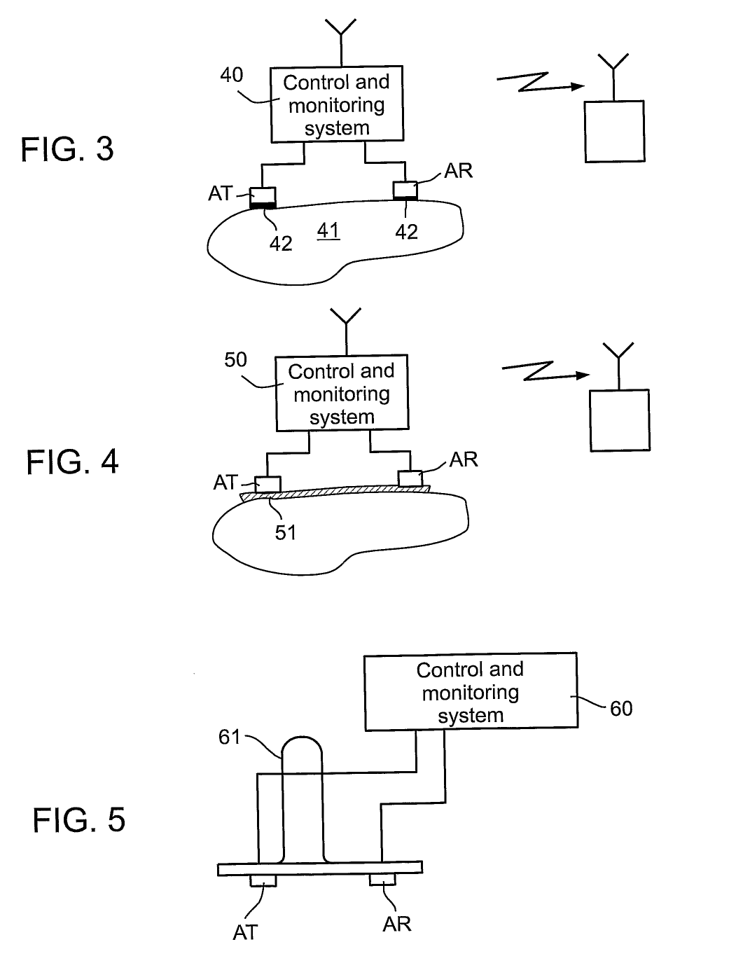

FIG. 3 schematically illustrates one arrangement which may be used in the apparatus of FIGS. 1 and 2 for attaching the acoustic transmitter AT and acoustical receiver AR of the sensor to the liver (or other viscus) 41. In this arrangement, they are attached by adhesive, shown schematically at 42. The control and monitoring system, indicated by block 40, may be constructed and operated in the same manner as described above with respect to FIGS. 1 and 2.

FIG. 4 schematically illustrates another attaching arrangement. In this arrangement, the acoustical transmitter AT and acoustical receiver AR of the sensor are carried by a flexible membrane 51, such as of silicone rubber or other elastomer, attached to the liver or other tissue adjacent to the liver. The acoustical transmission channel between the transmitter and receiver also includes a part of the liver, and therefore the transit time of the acoustical waves of the transmitter to the receiver enables monitoring any parameter affecting that transit time, as described above.

FIG. 5 schematically illustrates a further modification wherein the sensor is not to be implanted in the body, but rather is to be used for monitoring a parameter of the liver (or other viscus) during a surgical procedure and then to be removed from the body. For this purpose, the acoustical transmitter AT and acoustical receiver AR of the sensor are carried at one end of an implement 61, the opposite end of which serves as a handle for manipulating the sensor. The feedback electronics 60 in the apparatus illustrated in FIG. 5 would be carried by the implement 61, or connected to the sensor of the implement externally of the body, but would otherwise be constructed as described above with respect to FIGS. 1 and 2.

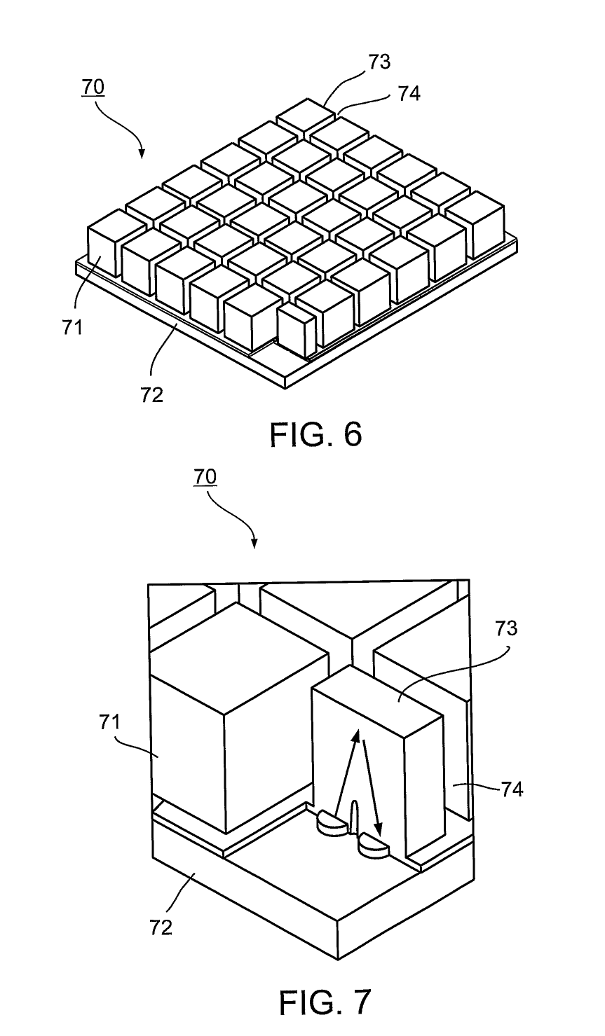

FIGS. 6 and 7 illustrate a particular construction of the sensor useful with respect to the implement illustrated in FIG. 5. Such a sensor 70 could include a plurality of acoustical transmitters (AT, FIG. 7) and a plurality of acoustical receivers AR defining an acoustical transmission channel ACT between each transmitter and each receiver, as shown particularly in FIG. 7, arranged in the form of a matrix as shown in FIG. 6. Such a matrix-type sensor 70 includes a plastic body 71 of a flexible elastomeric material, such as a silicone resin, having high acoustical transmissivity carried by a base layer 72, such as rubber, having low acoustical transmissivity. The high-acoustical transmissivity body 71 is divided into a plurality of cells 73 arranged in a waffle or an egg-crate configuration by a plurality of slots 74. Each such cell includes, as shown in FIG. 7, an acoustical transmitter AT and an acoustical receiver AR embedded in plastic body 71 adjacent to base layer 72, defining an acoustical channel ACT between them.

The matrix-type sensor illustrated in FIGS. 6 and 7 may thus be used for monitoring concurrently a plurality of different parameters of the internal organ of interest. It may also be used, for example, for monitoring changes in configuration or flatness of the organ or of any particular tissue, such as a bone section, or for measuring load distribution in a knee joint implant.

### Passive Wireless Sensor Constructions

The foregoing constructions use wireless sensors of the active type, i.e., those including their own power supplies and much of the electronics control circuitry. There are many applications, however, where active-type wireless sensors cannot be used because of the frequent need to re-charge or change the power supplies. In addition, an active sensor including its own power supply increases the volume of the sensor unit, which is generally not desirable in an implant operation.

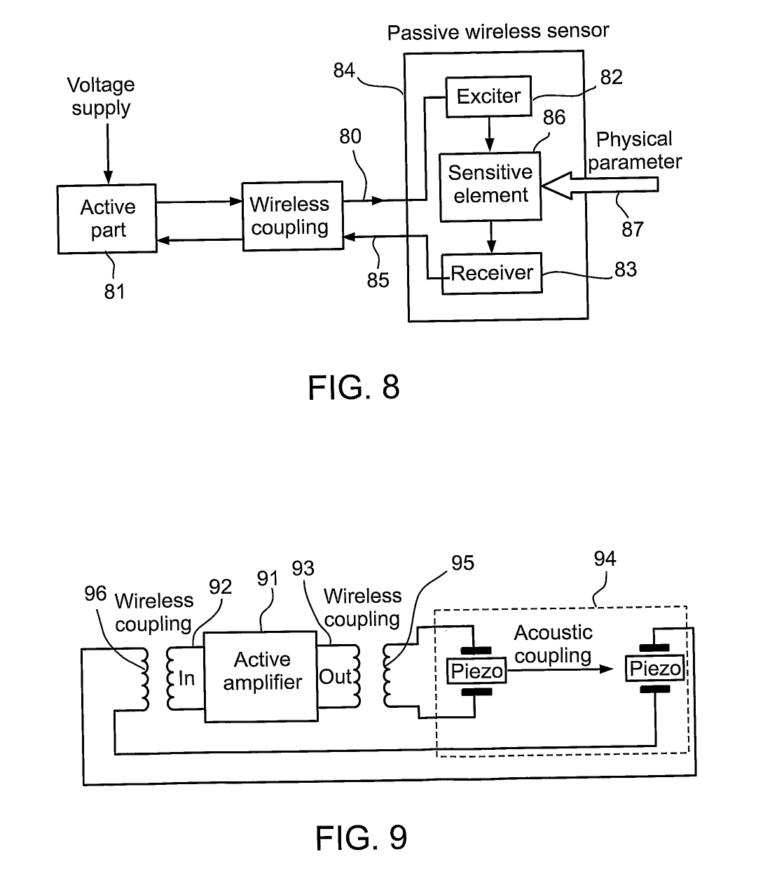

FIGS. 8-19 illustrate various types of passive wireless sensors that can be used in accordance with the present invention. In these illustrated systems, the sensor implanted in the body is a passive unit not including a power supply, whereas the monitoring of the transit time is effected by an active control unit which is located externally of the body.

The system illustrated in FIG. 8 includes a wireless coupling 80 for supplying power from the external active unit 81, to the exciter 82 and receiver 83 of the passive sensor unit 84, and a wireless coupling feedback circuit 85 for enabling the acoustical receiver 83 to control the acoustical transmitter of the sensor unit. The acoustical exciter is the transmitter, it being appreciated that the terms “transmitter” and “exciter” are alternative terms meaning the same in the context of this description.

The active part 81 of the system, located externally of the body, provides the energy to the implanted passive sensor unit 84, to actuate the exciter (transmitter) 82 to generate and transmit acoustical waves, via the acoustical transmission channel including the sensitive element or body 86 exhibiting the parameter of interest 87, to the receiver. It also supplies the energy to the receiver to control the exciter via the externally—located active part. The received energy returns to the active part 81 and is further amplified in order to control the passive sensor unit 84. Because of the feedback loop, a frequency is established in the system through the acoustical transmission channel of the passive sensor unit 84, which frequency depends on the physical parameter of interest 87 in the part of the body 86 within the acoustical channel between the exciter 82 and the receiver 83.

The wireless coupling between the active external unit 81 and the passive implanted sensor unit 84 may include a simplex transmission in each direction, or a duplex transmission in two directions. A duplex arrangement may include transmissions in two different frequencies, or may include an antisidetone circuit for isolating the transmission in one direction from the transmission in the other direction.

In addition, the wireless coupling may be effected in a two-port mode with respect to the passive sensor unit, in which the sensor unit has one input port and one output port. Alternatively, the wireless coupling may be effected in a one-port mode, in which the passive sensor unit acts like a variable impedance having a resonance depending on its physical parameters.

FIG. 9 shows a passive wireless sensor with simplex inductive couplings. The active part 91 contains the amplifier and two coils 92, 93 that are placed in different locations and have very low mutual inductance. The wireless sensor unit 94 has two coils 95, 96 as well. At the time of measurement these coils may be placed near the coils 92, 93 of the active part providing sufficient inductive coupling between the active unit 91 and the passive wireless sensor unit 94.

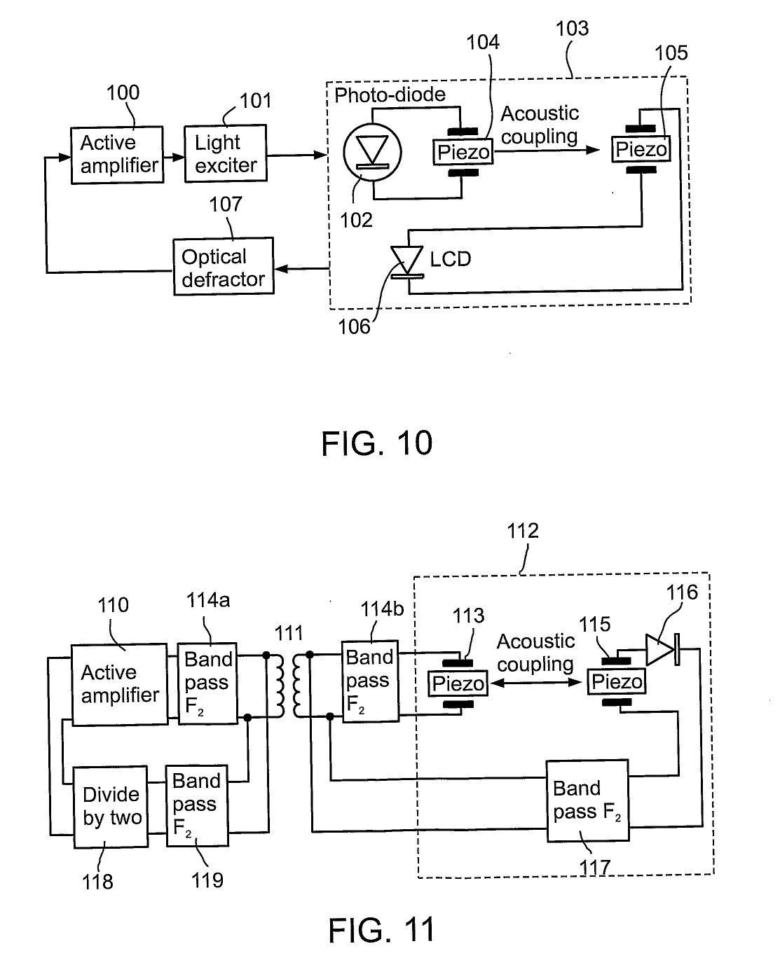

FIG. 10 shows the simplex light coupling for remote sensing. The output of the active amplifier part 100 is loaded with the light exciter 101, e.g. a laser or another powerful source of light. The pulses of light are transmitted as a narrow beam received by the photodiode 102 of the passive sensor unit 103, and are transformed to the ultrasound waves by the exciting piezo 104. The receiving piezo 105 is loaded with a light-emitting diode 106 that will excite the light only at the positive half-cycles of the received ultrasound waves. These light pulses are detected by a very sensitive optical detector 107 in the active part 104, to thereby provide a feed back loop for modulating the frequency.

Another alternative of the wireless coupling is the duplex transmission. In this case the same coupling is used for signal transmission in both directions, but special measures are necessary to split the transmitted and received signals on both ends of the coupling.

One method is to split the signals using two different frequencies. FIG. 11 shows the remote feed back with frequency multiplication on the side of the passive sensor unit 113 and frequency division on the side of the active amplifier unit 110. The active amplifier unit sends the frequency F₁ to the passive sensor unit 112 via the inductive coupling and band-pass filter 114a, and the frequency comes to the exciting piezo 113 via the band-pass filter 114b. The signals from the receiving piezo 115 are rectified by a diode 116; thus its frequency is multiplied by two (F₂=2·F₁). The frequency F₂ comes to the same coil of the sensor via a band-pass filter 117, and further it passes through coupling without interference with the frequency F₁. The frequency F₂ passes from the coil of the active unit 110 to a divider 118 via the band-pass filter 119. The divided frequency (F₂/2=F₁) comes to the input of the active amplifier unit 110, thereby completing the feed back.

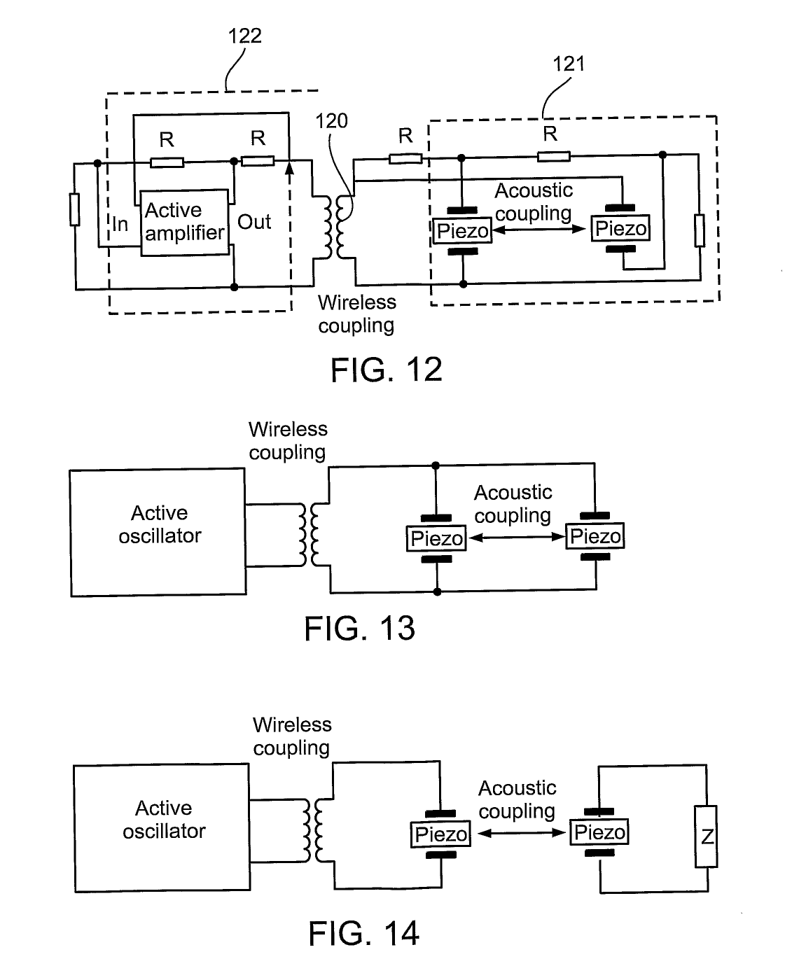

Another method for duplex transmission is by using an antisidetone circuit as shown in FIG. 12. The loads Z and the corresponding coils 120 have the same impedance; voltage drops are thus created in the resistors R with the same amplitude and in the opposite direction. Thus the influence of the output voltage to the input is eliminated. However, the signal from the passive sensor unit 121 is applied to the input of the active amplifier unit 122. The splitting of the signals on the sensor side is performed analogously.

Another possibility for single coupling is by using a passive wireless sensor in the one-port mode. FIGS. 10-12 illustrate the use of the sensor in the two-port mode, wherein there is one input port and one output port in the sensor. In other words, one piezo works as the exciter and the second piezo works as the receiver of the ultrasound waves. The sensor passive unit may be described as a device with certain transform functions. On other hand, the active unit has the input and output ports as well.

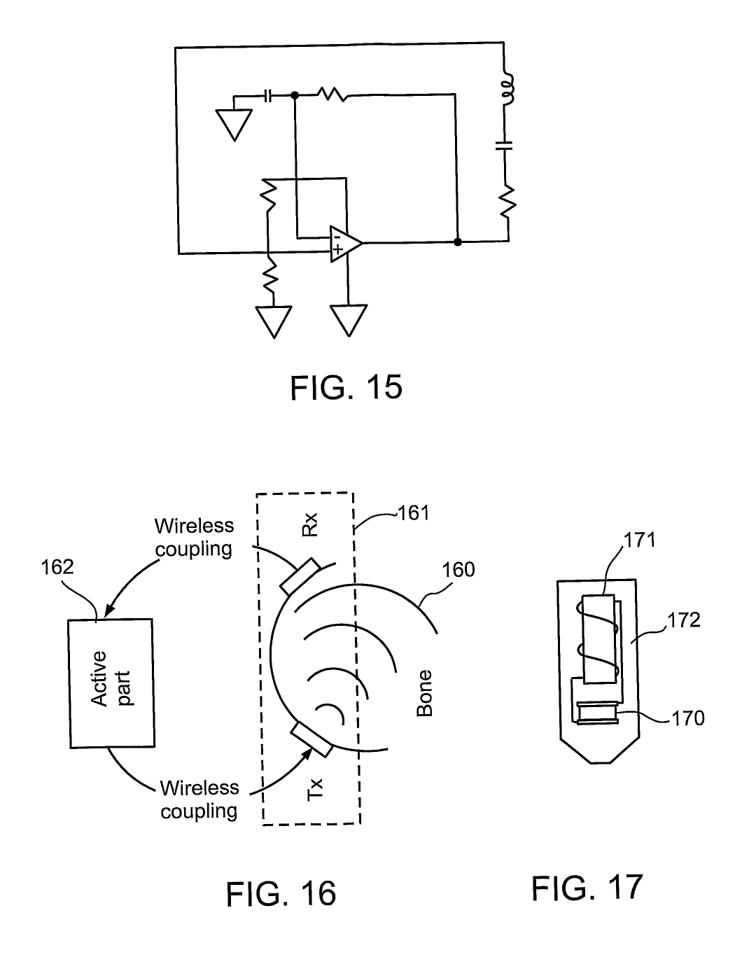

However, it is possible to use a one port configuration in both the active unit and the sensor passive unit. In this case the sensor works rather like the impedance which has a resonance depending on the physical parameter. FIGS. 13 and 14 show various connections of the piezos, and various configurations of the oscillator, e.g. as shown on FIG. 15.

FIGS. 16 and 17 illustrate the apparatus for measuring changes in bone density. The bone 160 itself is used as the sensitive element of the passive sensor unit 161. The changes of bone density result in changes in the ultrasound velocity, from the active unit 162 through the bone and, thereby, changes in the transit time; consequently the resulting frequency is a measure of the bone density.

FIG. 17 illustrates a transducer construction in detail. Each transducer is built as a separate unit that contains the piezo 170 and the coupling coil 171 embedded in the housing 172. It provides the possibility to place the transducers in various locations.

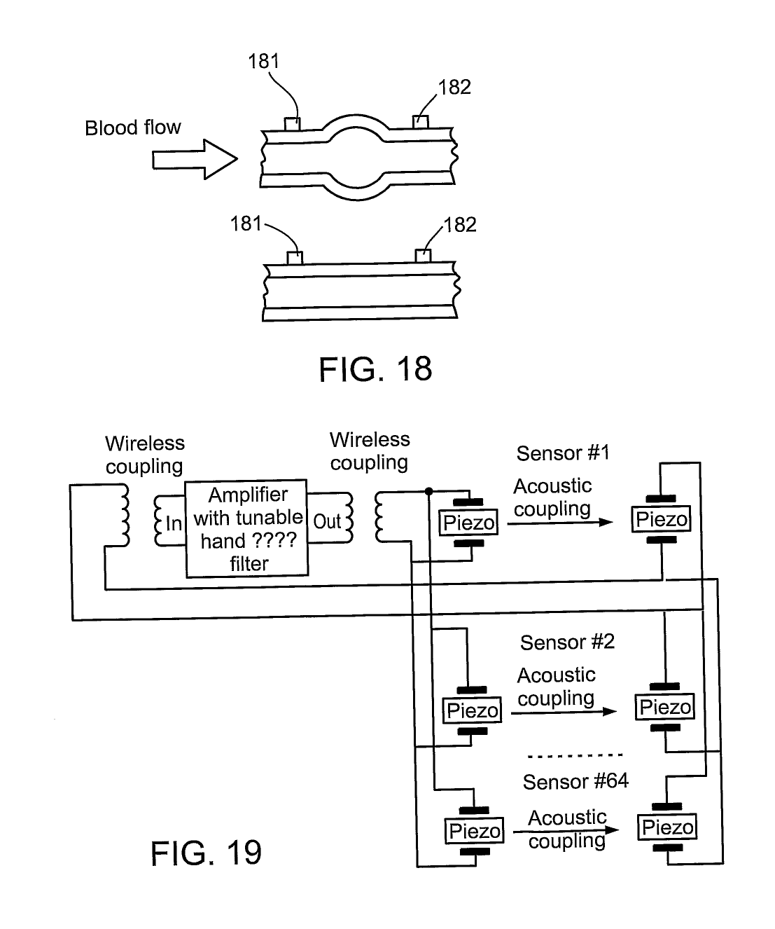

FIGS. 18a and 18b illustrate a sensor for measuring the flexibility of blood vessels. Each heart beat results in increasing the pressure of the blood in the vessel to generate a pulse of high pressure as shown in FIG. 18a. Such pulses propagate along the vascular system with relatively low speed, about 5-10 m/sec. The vessel's wall is used as the sensitive element. The changes in its flexibility result in changes in the transit velocity of the pulse waves through the blood vessel between the two piezos 181, 182, and consequently changes in the resulting frequency. In addition, the changes of pressure at the time of systole (FIG. 18a) and diastole (FIG. 18b) will change the transit distance between the piezos. This information may be used to measure blood pressure and heart rate.

When the matrix type sensor illustrated in FIGS. 6 and 7 is used, each pair of piezos works according to the wireless technology as described above; thus every sensor unit measures the displacement of its own top surface with high resolution. When pressing a bone section to the elastomeric surface 71 of the sensor unit, surface 71 conforms to the shape of the bone section.

In order to prevent the interference between simultaneously working sensors, each pair has the specific frequency range of its piezos. The active unit contains the tunable band-pass filter. When the filter is tuned to the frequency range of the specific pair, this pair only may provide wireless feed back. Consequently, the resulting frequency depends on the displacement of the specific sensor that is excited by the active part in this time. To know the distribution of the force on the matrix area the frequency range of the filter is scanned along the full band of the device.

FIG. 19 illustrates a system including a plurality of passive sensors, such as arranged in a matrix, as described above.

It will be appreciated that the same apparatus and method may be used in non-medical applications as well, e.g. for checking water seepage in concrete building constructions.

While the invention has been described with respect to several preferred embodiments, it will be appreciated that these are set forth merely for purposes of example, and that many other variations, modifications and applications of the invention may be made.

1. A method of monitoring a predetermined parameter of interest in a body, comprising:
applying to said body a sensor including an acoustical transmitter and an acoustical receiver spaced from the transmitter to define between them an acoustical transmission channel through a part of the body exhibiting the parameter of interest;
and monitoring from a remote location the transit time of an acoustical wave transmitted through said acoustical transmission channel from said acoustical transmitter to said acoustical receiver, said monitored transit time being influenced by the parameter of interest and thereby providing a measurement of said parameter of interest.

2. The method according to claim 1, wherein said sensor is implanted in said body and includes an RF transmitter for transmitting its measurements to a receiver externally of said body.

3-11. (canceled)

12. The method according to claim 1, wherein said body is an organ of a living body, and said sensor is applied to said body to monitor said parameter in the course of or following a surgical procedure.

13-19. (canceled)

20. Apparatus for monitoring a predetermined parameter of interest in a body, comprising:
a sensor including an acoustical transmitter and an acoustical receiver constructed to be applied within the body to define an acoustical transmission channel through a part of said body exhibiting the parameter of interest;
and a control and monitoring system to be located at a remote location from said sensor for actuating said acoustical transmitter to transmit acoustical waves through said acoustical transmission channel, for actuating said acoustical receiver to receiver said acoustical waves, and for monitoring the transit time of transmission of the transmitted waves from the acoustical transmitter to the acoustical receiver, to provide a measurement of said predetermined parameter of interest.

21. The apparatus according to claim 20, wherein said sensor is constructed as a unit for implantation in said body and includes an RF transmitter for transmitting its measurements to a receiver externally of said body.

22. The apparatus according to claim 21, wherein said sensor unit is a passive unit; and said control and monitoring system is in an active unit to be located externally of said body which includes a wireless coupling for supplying power to the sensor unit, and a wireless coupling for a feedback circuit for enabling receiver to control said transmitter of the sensor unit.

23. The apparatus according to claim 21, wherein said wireless couplings are inductive couplings.

24. The apparatus according to claim 21, wherein said wireless couplings are optical couplings.

25. The apparatus according to claim 21, wherein said wireless couplings include a simplex transmission in each direction.

26. The apparatus according to claim 21, wherein said wireless couplings include a duplex transmission in two directions.

27. The apparatus according to claim 26, wherein said duplex transmission includes transmission in two different frequencies.

28. The apparatus according to claim 26, wherein said duplex transmission includes an antisidetone circuit for isolating the transmission in one direction from that in the other direction.

29. The apparatus according to claim 21, wherein said wireless coupling is effected in a two-port mode with respect to the sensor unit, in which the sensor unit has one input port and one output port.

30. The apparatus according to claim 21, wherein said wireless coupling is effected in a one-port mode, in which the sensor acts like a variable impedance having a resonance depending on its physical parameters.

31. (canceled)

32. The apparatus according to claim 21, wherein said sensor includes a membrane carrying said acoustical transmitter and said acoustical receiver and attachable to an organ in said body, or to tissue adjacent to said organ.

33. The apparatus according to claim 21, wherein said sensor is carried at one end of an implement having a handle at the opposite end for manipulating the sensor.

34. (canceled)

35. The apparatus according to claim 21, wherein said sensor includes a plurality of acoustical transmitters and a plurality of acoustical receivers defining a plurality of acoustical transmission channels arranged in the form of a matrix.

36. The apparatus according to claim 35, wherein said matrix of acoustical transmitters and acoustical receivers are embedded in a plastic body having high acoustical transmissivity, said plastic body being joined on one face to a layer of material having low acoustical transmissivity.

37. The apparatus according to claim 36, wherein the opposite face of said plastic body, facing away from said layer of material have low acoustical transmissivity, is formed with slots of egg-crate configuration tending to isolate said plurality of acoustical transmission channels from each other.

38. The apparatus according to claim 21, wherein said transit time is monitored by changes in the frequency of the acoustical waves transmitted by said transmitter to said receiver via said acoustical transmission channel, while maintaining the number of waves in a loop including said acoustical transmission channel as a whole integer irrespective of changes in said predetermined parameter of interest.
